(19) United States
(12) Patent Application Publication
(10) Pub. No.: US 2024/0366186 A1
(43) Pub. Date: Nov. 7, 2024

(54) APPARATUS, SYSTEMS AND METHODS FOR PERFORMING WIDE FREQUENCY RANGE ULTRASOUND IMAGING USING A SINGLE ULTRASOUND PROBE

(71) Applicant: Clarius Mobile Health Corp., Vancouver (CA)

(72) Inventors: Daniel RAHARDJA, Burnaby (CA); Binda ZHANG, Surrey (CA); Kris DICKIE, Vancouver (CA)

(21) Appl. No.: 18/142,575

(22) Filed: May 2, 2023

(51) Int. Cl. A61B 8/00

(52) U.S. Cl. CPC A61B 8/4477; A61B 8/4488; A61B 8/4494

(57) ABSTRACT

An ultrasound probe comprising a body comprising an imaging end and a non-imaging end, a first array comprising a first plurality of transducer elements disposed on the imaging end for forming a first beam type from a first transmit surface, a second array comprising a second plurality of transducer elements disposed on the imaging end, for forming a second beam type from a second transmit surface, wherein each the first array and the second array are longitudinally adjacent to each other and the first transmit surface and second transmit surface are separate, a circuit connected to the first plurality of transducer elements and the second plurality of transducer elements, wherein the circuit is capable of activating a number of elements equal to a total of the first plurality of transducer elements and the second plurality of transducer elements and wherein such activation is mutually exclusive.

### FIELD

The present disclosure relates generally to ultrasound imaging, and in particular, methods, systems and apparatus that enable wide frequency range ultrasound imaging using a single ultrasound transducer.

### BACKGROUND

Ultrasound imaging has a wide range of medical applications. For example, ultrasound imaging provides a relatively fast and non-invasive way to assess abdominal organs such as the bladder, liver, uterus, kidneys, and the like. Ultrasound imaging may also be used to obtain images of the heart and vascular structures. Ultrasound users often require several different types of transducers to cover this variety of imaging applications. These transducers vary in several parameters such as bandwidth, center frequency, array dimensions, and curvature. Higher center frequency provides improved axial resolution at the expense of depth of penetration whereas a lower center frequency provides greater penetration but poorer axial resolution. Larger arrays provide improved field of view but may not be appropriate for areas of the body with limited soft-tissue access such as through the rib cage. An array curvature provides a wider field of view in the far-field as compared to a linear array which has the same lateral resolution in both the near field and far field.

With all of this in mind, traditional ultrasound systems are typically used with a number of different ultrasound probes that are designed to image different parts of the body. Ultrasound probes (also called ultrasound transducers or scanners) generally contain a number of transducer elements that can be selectively pulsed to generated ultrasound signals. These ultrasound signals are projected into a volume of tissue and corresponding echo signals are processed to generate an ultrasound image. Different types of ultrasound probes have different transducer element configurations to allow for imaging different parts of the body.

For example, a phased-array probe typically has a small footprint containing a small number of transducer elements positioned on the probe head. The small footprint allows the probe to be positioned on parts of the body that have constricted space. To obtain a sufficiently wide field of view using the small number of transducer elements on the probe head, the ultrasound signals are steered in many different directions during multiple phases when projected into the volume of tissue being imaged. The phased multi-directional steering of a phased-array probe makes it suitable for imaging the heart because the ultrasound signals can be projected through the intercostal space in between a patient's ribs. Generally, phased-array probes have lower frequency ranges (e.g., 1-5 Mhz) to allow for sufficient penetration to capture cardiac images.

In another example, a linear probe is generally designed for superficial imaging. The crystals are aligned in a linear fashion within a flat head and produce sound waves in a straight line. This probe has higher frequencies (5-13 MHz), which provides better axial resolution but less deep penetration.

Further, convex transducers (also called curved linear array) are typically used in abdominal and pelvic sonography. Crystals are arranged next to each other along a curved (convex) surface which enables a widened field of view, especially in the depth display, but also assures good near field resolution. The resulting image is curved-shaped with the diameter increasing with the depth. Curved transducers with a small aperture and wide scanning field are available for transcutaneous, intraoperative, and intracavitary use.

When examining a patient, an ultrasound operator may need to switch probes during the examination (from phased-array to convex to linear) in order to complete the examination (for example, to examine the heart with a phased-array probe, carotid artery with a linear probe and the general abdomen with a curved linear-array probe). With conventional systems, switching probes typically involves physically removing one probe from an ultrasound machine, plugging in a different probe, and operating one or more controls on the ultrasound machine to cause the ultrasound machine to operate in the desired imaging mode that works with the newly attached probe. This can be time consuming and can present problems in certain medical environments such as critical emergency care.

Attempts have been made to address this problem by devising an ultrasound probe with a separate transducer at each end of the probe. There are a number of functional drawbacks and inefficiencies in these product offerings, including but not limited to the challenge of sterility and cleanliness. Since the coupling medium (ultrasound gel) is spread across both ends of the probe as it is flipped from one end to the other during use, containment of the medium is impossible and practical usage is compromised as the grip/centre portion of the probe can become covered in coupling medium.

There is thus a need improved methods and apparatus for imaging different areas of a patient without the need to switch between different probes. This need is pronounced in (although not exclusive to) emergency care environments. The embodiments discussed herein may address and/or ameliorate at least some of the aforementioned design considerations identified above. The foregoing examples of the related art and limitations related thereto are intended to be illustrative and not exclusive. Other limitations of the related art will become apparent to those of skill in the art upon a reading of the specification and a study of the drawings herein.

### BRIEF DESCRIPTION OF THE DRAWINGS

Non-limiting examples of various embodiments of the present disclosure will next be described in relation to the drawings, in which:

FIG. 1 shows a perspective view of an ultrasound probe for performing wide frequency imaging, in accordance with at least one aspect of the present invention;

FIG. 2 shows a top plan view of an ultrasound probe for performing wide frequency imaging, in accordance with at least one aspect of the present invention;

FIG. 3 shows a top plan view of the ultrasound probe for performing wide frequency imaging, of FIG. 2, without the acoustic lensing material;

FIG. 4 shows a partial cut-away elevation perspective view of an ultrasound probe for performing wide frequency imaging, in accordance with at least one aspect of the present invention;

FIG. 5 shows a top plan view of the ultrasound probe of FIG. 2, with the acoustic lensing material over each transducer array but exposing flexible PCB and other electronic architecture under each transducer array;

FIG. 6 shows a side view in partial cut-away of an ultrasound probe for performing wide frequency imaging,, in accordance with at least one aspect of the present invention, exposing both a linear transducer stack and a phased-array transducer stack, including flexible PCB and other electronic architecture under each transducer array;

FIG. 7 shows is a functional block diagram of an ultrasound probe for performing wide frequency imaging, according to certain embodiments of the present disclosure;

FIG. 8 shows an illustration of i) an ultrasound probe for performing wide frequency imaging, in accordance with at least one aspect of the present invention in operation with a phased array stack activated and scanning a chest region; and ii) a multi-purpose display device in wireless communication with the ultrasound probe and showing on a screen an ultrasound image of a heart;

FIG. 9 shows an illustration of i) an ultrasound probe for performing wide frequency imaging, in accordance with at least one aspect of the present invention in operation with a linear stack activated and scanning a neck region; and ii) a multi-purpose display device in wireless communication with the ultrasound probe and showing on a screen an ultrasound image of a carotid artery;

FIG. 10 shows a schematic diagram of various components of an ultrasound probe for performing wide frequency imaging, according to an embodiment of the present invention;

FIG. 11 shows a schematic diagram of a geometry of a transducer stack with dimensional guide references around the piezoelectric array;

FIG. 12A shows a mold at one step of a process to form a dual stack transducer according to an embodiment of the present invention; and

FIG. 12B shows a mold at a further step (to that shown in FIG. 12A) of a process to form a dual stack transducer according to an embodiment of the present invention.

### DETAILED DESCRIPTION OF THE INVENTION

### A. Glossary

The term “anatomical feature” means any part of a human body, an animal, or phantom, and may refer to an entire organ, a part of an organ, damage to an organ, abnormality of an organ, illness, an unwanted growth, and the like. In obstetric practise, it may refer to an entire fetus, part of a fetus, or an entire organ, a part of an organ, damage to an organ, abnormality of an organ, illness, an unwanted growth, and the like.

The terms “dual array probe/dual array scanner/dual array ultrasound probe” may be used interchangeably with “wide frequency ultrasound probe/wide frequency scanner/wide frequency ultrasound probe” in describing the probe of the present invention, as it is understood that the wide frequency range of scanning of the probe of the present invention is afforded by the arrangement of dual arrays, within the imaging end of the probe.

The term “focusing” when used with respect to an ultrasound beam refers to a method of creating a narrow point in the cross-section of the ultrasound beam called the focal point. It is at the focal point where the lateral resolution of the beam is the greatest also. Before the focal point is the near field or Fresnel zone, where beams converge and distal to this focal point is the far field or Fraunhofer zone where the beams diverge.

The term “communications network” can include both a mobile network and data network without limiting the term's meaning, and includes the use of wireless (e.g. 2G, 3G, 4G, 5G, WiFi™, WiMAX™, Wireless USB (Universal Serial Bus), Zigbee™, Bluetooth™ and satellite), and/or hard wired connections such as local, internet, ADSL (Asymmetrical Digital Subscriber Line), DSL (Digital Subscriber Line), cable modem, T1, T3, fiber-optic, dial-up modem, television cable, and may include connections to flash memory data cards and/or USB memory sticks where appropriate. A communications network could also mean dedicated connections between computing devices and electronic components, such as buses for intra-chip communications.

The term “imaging contact surface”, as used herein, refers to the surface of the wide frequency probe which engages a matter to be scanned (such matter for example being skin of a patient). In one embodiment, the imaging contact surface may be a unitary lens material which covers both the first transducer stack and the second transducer stack.

The term “module” can refer to any component in this invention and to any or all of the features of the invention without limitation. A module may be a software, firmware or hardware module, and may be located, for example, in the dual function ultrasound scanner and/or in any associated electronic device to which it is capable of communicating.

The term “multi-purpose electronic device” or “display device” is intended to have broad meaning and includes devices with a processor communicatively operable with a screen interface, for example, such as, laptop computer, a tablet computer, a desktop computer, a smart phone, a smart watch, spectacles with a built-in display, a television, a bespoke display or any other display device that is capable of being communicably connected to an ultrasound scanner. Such a device may be communicatively operable with an ultrasound scanner and/or a cloud-based server (for example via one or more communications networks).

The term “operator” (or “user”) may refer to the person that is operating an ultrasound scanner (e.g., a clinician, medical personnel, paramedical personnel, a sonographer, ultrasound student, ultrasonographer and/or ultrasound technician).

The term “element” may be used interchangeably with “piezoelement”, and “piezoelectric element” refers to any crystal/ceramic element with piezoelectric properties, which is usually although not exclusively, lead zirconate titanate (PZT). Generally, element thickness is determined by a desired and selected resonance frequency wherein a thicker element produces a lower frequency oscillation while a thinner element produces a higher frequency oscillation.

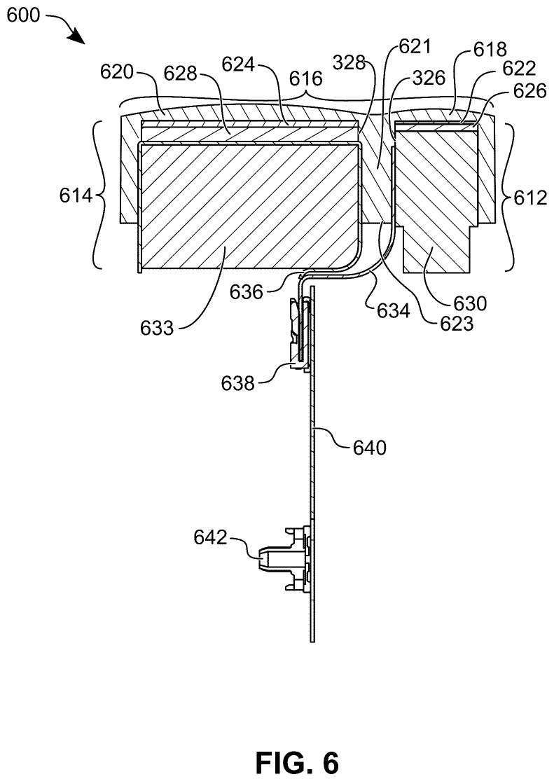

The term “longitudinally adjacent” is a spatial descriptor describing the relative positions, within the wide frequency probe of the invention, of one or more of i) the first array and the second array and ii) the first transducer stack and the second transducer stack. The nature of the relative orientation of the dual stacks may be seen best in FIGS. 3 and 6.

The term “processor” can refer to any electronic circuit or group of circuits that perform calculations, and may include, for example, single or multicore processors, multiple processors, an ASIC (Application Specific Integrated Circuit), and dedicated circuits implemented, for example, on a reconfigurable device such as an FPGA (Field Programmable Gate Array). A processor may perform the steps in the flowcharts and sequence diagrams, whether they are explicitly described as being executed by the processor or whether the execution thereby is implicit due to the steps being described as performed by the system, a device, code or a module. The processor, if comprised of multiple processors, may be located together or geographically separate from each other. The term includes virtual processors and machine instances as used in cloud computing or local virtualization, which are ultimately grounded in physical processors.

The term “system” when used herein, and not otherwise qualified, refers to a system for scanning using both high frequencies and low frequencies using separate transducer stacks, both situated at one “scanning” or “operational” end of a probe, the system being a subject of the present invention. In various embodiments of the present invention, the system may include, but is not limited to such (i) a wide frequency scanner (with a display device), and/or (ii) a wide frequency ultrasound scanner, display device and a server. In other embodiments, a system refers to any of the above-noted components in association with any medical interventional tool assisted by ultrasound, including but not limited to, a needle.

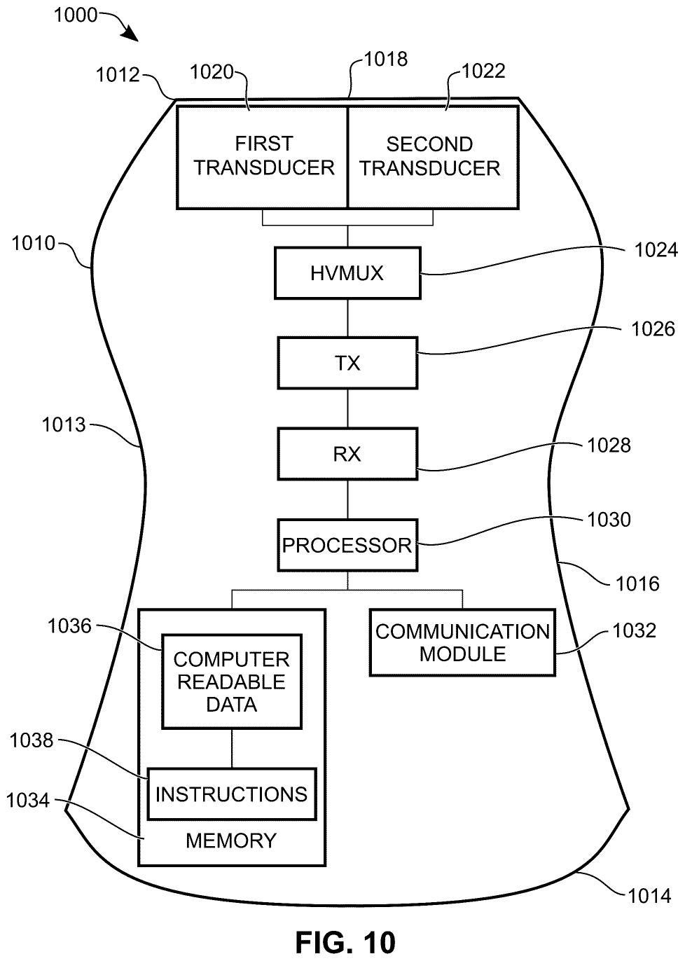

The term “transducer stack” or “ultrasound transducer stack” as used herein comprises generally crystal/ceramic element with piezoelectric properties, positive and ground electrodes on the faces of the element, damping (backing) block which is adhered in some manner to the back of the crystal (behind the positive electrode), and at least one matching layer. In an embodiment of the invention, as illustrated best in FIG. 6 and FIG. 10, a wide frequency probe comprises a first complete transducer stack for the “first array” and a second complete transducer stack for the “second array”. Thereover and therebetween the first transducer stack and the second transducer stack (e.g., as may be provisioned during manufacturing) is an acoustic lens material shown as 620, 621 and 625 in FIG. 6.

The term “transmit surface” as used herein refers to either or a combination of a piezoelectric array (first and/or second) together with its corresponding matching layer and/or just the matching layer (for example first matching layer and/or second matching layer). The matching layers are important acoustic materials in an ultrasound transducer stack and are used to propagate as much ultrasound energy as possible, by matching a target medium with the energy-creating piezoelectric array (first and/or second). With the scope of the present invention, each of the first transducer stack and the second transducer stack comprises a respective matching layer and hence transmit surface (first transmit surface and second transmit surface). These surfaces may, in some aspects, be generally planar contact surfaces for contact with a subject/patient.

The term “ultrasound imaging apparatus” when used herein, and not otherwise qualified may be used interchangeably with various other terms, such as, for example, ultrasound probe, wide frequency probe, wide frequency scanner, ultrasound scanner, ultrasound transducer, ultrasound imaging device, scanner, probe, or ultrasound image capturing device.

The term “ultrasound image frame” (or “image frame” or “ultrasound frame”) refers to a frame of post-scan conversion data that is suitable for rendering an ultrasound image on a screen or other display device.

For simplicity and clarity of illustration, where considered appropriate, reference numerals may be repeated among the figures to indicate corresponding or analogous elements or steps. In addition, numerous specific details are set forth in order to provide a thorough understanding of the exemplary embodiments described herein. However, it will be understood by those of ordinary skill in the art that the embodiments described herein may be practiced without these specific details. In other instances, certain steps, signals, protocols, software, hardware, networking infrastructure, circuits, structures, techniques, well-known methods, procedures and components have not been described or shown in detail in order not to obscure the embodiments generally described herein.

Furthermore, this description is not to be considered as limiting the scope of the embodiments described herein in any way. It should be understood that the detailed description, while indicating specific embodiments, are given by way of illustration only, since various changes and modifications within the scope of the disclosure will become apparent to those skilled in the art from this detailed description. Accordingly, the specification and drawings are to be regarded in an illustrative, rather than a restrictive, sense.

### B. Exemplary Embodiments

This description is not to be considered as limiting the scope of the embodiments described herein in any way. It should be understood that the detailed description, while indicating specific embodiments, are given by way of illustration only, since various changes and modifications within the scope of the disclosure will become apparent to those skilled in the art from this detailed description. Accordingly, the specification and drawings are to be regarded in an illustrative, rather than a restrictive, sense.

By way of background, the system and method of the present invention uses transducers (piezoelectric or capacitive devices operable to convert between acoustic and electrical energy) to scan a planar region or a volume of an anatomical feature. Electrical and/or mechanical steering allows transmission and reception along different scan lines wherein any scan pattern may be used. Ultrasound data representing a plane or volume is provided in response to the scanning. The ultrasound data is beamformed, detected, and/or scan converted. The ultrasound data may be in any format, such as polar coordinate, Cartesian coordinate, a three-dimensional grid, two-dimensional planes in Cartesian coordinate with polar coordinate spacing between planes, or other format. The ultrasound data is data which represents an anatomical feature sought to be assessed and reviewed by a sonographer.

The embodiments herein allow for the provision of an ultrasound probe, ultrasound systems and ultrasound-based methods to enable scanning using two different arrays (a first and a second), each comprising a plurality of transducer elements, and both situated on an imaging end of one, singular ultrasound probe. In this way, a user can scan using each array without requiring multiple probes be substituted and used during the scanning process and image acquisition. within a single probe. The ultrasound probe, ultrasound systems and ultrasound-based methods of the present invention are particularly (although not exclusively) useful in emergency and quick response triage type settings where it is desirable to quickly and efficiently scan various anatomical features in succession, without switching probes, powering up a new probe, changing settings, applying conductive agent to new probe, etc. In such settings where time can be the difference between life and death, and the rapid assessment of more than one anatomical feature of a patient is required, the wide frequency probe, ultrasound systems and ultrasound-based methods of the present invention offer significant practical advantages.

In one aspect of the present disclosure, there is provided an ultrasound probe comprising a body comprising an imaging end and a non-imaging end, a first array comprising a first plurality of transducer elements disposed on the imaging end, a second array comprising a second plurality of transducer elements also disposed on the imaging end, wherein each the first array and the second array are longitudinally adjacent to each other, a circuit connected to the first plurality of transducer elements and the second plurality of transducer elements; wherein the circuit is capable of activating, in a mutually exclusive manner, the first plurality of transducer elements and the second plurality of transducer elements.

In another broad aspect of the present disclosure, there is provided an ultrasound imaging system comprising an ultrasound probe configured to provide image data, a processor configured to assemble images from said image data and a display configured to display said images, wherein said ultrasound probe comprises a body comprising an imaging end and a non-imaging end, a first array comprising a first plurality of transducer elements disposed on the imaging end, a second array comprising a second plurality of transducer elements also disposed on the imaging end, wherein each the first array and the second array are longitudinally adjacent to each other, a circuit connected to the first plurality of transducer elements and the second plurality of transducer elements; wherein the circuit is capable of activating, in a mutually exclusive manner, the first plurality of transducer elements and the second plurality of transducer elements.

In another broad aspect of the present disclosure, there is provided an ultrasound imaging method comprising, by a wide frequency ultrasound probe, imaging in a first mode using a first array comprising a first plurality of transducer elements disposed on an imaging end of the probe, by a circuit activating only the first plurality of transducer elements; switching, to a second mode different from the first mode, using a second array comprising a second plurality of transducer elements disposed on the imaging end of the probe, by the circuit activating only the second plurality of transducer elements; wherein the second array is longitudinally adjacent to the first array and wherein the circuit is capable of activating, in a mutually exclusive manner, the first plurality of transducer elements and the second plurality of transducer elements.

In some embodiments, there is provided an ultrasound probe comprising a body comprising an imaging end and a non-imaging end, a first array comprising a first plurality of transducer elements disposed on the imaging end for forming a first beam type from a first transmit surface, a second array comprising a second plurality of transducer elements disposed on the imaging end, for forming a second beam type from a second transmit surface, wherein each the first array and the second array are longitudinally adjacent to each other and the first transmit surface and second transmit surface are separate, a circuit connected to the first plurality of transducer elements and the second plurality of transducer elements; wherein the circuit is capable of activating a number of elements equal to a total of the first plurality of transducer elements and the second plurality of transducer elements and wherein such activation is mutually exclusive.

In some embodiments, there is provided an ultrasound imaging system comprising an ultrasound probe configured to provide image data, a processor configured to assemble images from said image data and a display configured to display said images, wherein said ultrasound probe comprises a body comprising an imaging end and a non-imaging end, a first array comprising a first plurality of transducer elements, a second array comprising a second plurality of transducer elements disposed on the imaging end, wherein each the first array and the second array are longitudinally adjacent to each other to each other, wherein the first plurality of transducer elements and the second plurality of transducer elements are connected to a circuit, wherein the circuit is capable of activating a number of elements equal to a total of the first plurality of transducer elements and the second plurality of transducer elements.

In some embodiments, there is provided an ultrasound imaging method comprising, by a dual array ultrasound probe, imaging in a first mode using a first array comprising a first plurality of transducer elements disposed on an imaging end of the probe, therein producing a first beam from a first transmit surface by a circuit activating only the first plurality of transducer elements; switching, to a second mode different from the first mode, using a second array comprising a second plurality of transducer elements disposed on the imaging end of the probe, therein producing a second beam from a second transmit surface by the circuit activating only the second plurality of transducer elements; wherein the second array is longitudinally adjacent to the first array and the first transmit surface and second transmit surface are separate; and wherein the circuit is capable of activating a number of elements equal to a total of the first plurality of transducer elements and the second plurality of transducer elements and wherein such activation is mutually exclusive.

In some embodiments, a circuit is communicatively coupled to both a mode selection input and to each of the first plurality of transducer elements and the second plurality of transducer elements, the circuit receiving mode selection input to activate a first mode, in which signals are transmitted only by the first plurality of transducer elements and a second mode in which signals are transmitted only by the second plurality of transducer elements. As such, in some embodiments, imaging in a first mode further includes pulsing groups of adjacent first plurality of transducer elements disposed on an imaging end of the probe wherein a second array comprising a second plurality of transducer elements disposed on the same imaging end of the probe are not pulsed.

In some embodiments, the first array produces a high frequency bandwidth, and the second array produces a low frequency bandwidth.

In some embodiments, the first array is linear, and the second array is a phased array.

In some embodiments, the first plurality of transducer elements and the second plurality of transducer elements are coupled to a common switching matrix having one or input lines and a one or more of output lines, the output lines being coupled to one of the transducer elements of the first plurality and the second plurality, the switching matrix coupled to a mode selection input and having a first mode in which the output lines coupled to the transducer elements of the first plurality is coupled to one of the input lines and a second mode in which one of the input lines is coupled to each of the input lines coupled to the transducer elements of the second plurality and pairs of the output lines coupled to adjacent transducer elements of the first plurality are each coupled to one of the input lines.

Other features of an ultrasound transducer probe which may be provided according to some aspects of the present application, and which may contribute to the ultrasound probe's versatility include the ultrasound probe's physical form and the architecture of the ultrasound probe circuitry.

In some embodiments, a first array comprising a first plurality of transducer elements and a second array comprising a second plurality of transducer elements are disposed in a common plane but separated by a colinear space.

In some embodiments, a first transducer stack and a second transducer stack are covered by a unitary lens material and are disposed in a common plane but separated by a colinear space. In other embodiments, a transmit surface and a second transmit surface are covered by a unitary lens material.

In some embodiments, the circuit is a multiplex circuit and activates the first plurality of transducer elements starting from a start element which is either 0 or 1 and activates the second plurality of transducer elements starting from a start element which is neither 0 nor 1.

In some embodiments, a pitch of the second array is substantially the same size as the pitch in the first array. Preferably, the pitch spacing of within each of the first array and the second array is between 200-260 microns, more preferably between 240 and 260 microns and even more preferably between 250-260 microns. In a most preferred embodiment, the first array is linear, and the second array is a phased-array, and the pitch of each array is 260 microns.

In some embodiments, a circuit is capable of activating a number of elements equal to a total of the first plurality of transducer elements and the second plurality of transducer elements and such total may in some embodiments be 192 elements. In an embodiment, the first array is linear, and the second array is a phased-array and the total number of transducer elements within the probe, which may be activated by the circuit is 192, wherein the first array and the second array is separated by a colinear space. Within the total number of transducer elements, a first subset of the total number of elements (e.g., in an embodiment with 192 total elements, from 64-80 elements) may be allocated to a second/phased array and a second subset of the total number of elements (e.g., in an embodiment with 192 elements, from 112-128) may be allocated to a first/linear array. For example, in a particular embodiment, a total of the first plurality of transducer elements and the second plurality of transducer elements is 192 which includes 112 which are activatable as the first plurality of transducer elements and 80 which are activatable as the second plurality of transducer elements.

In some embodiments, ultrasound signals generated by the second array are steered in a direction so as to create a sector (phased array) image. In some embodiments, the sector image has a sector angle up to 90 degrees.

In some embodiments, the dual array ultrasound probe comprises an enclosed body comprising an imaging end and a non-imaging end, and at the imaging end, a first transducer stack, a second transducer stack fully separated from the first transducer stack, and a colinear space therebetween, wherein the first transducer stack comprises a first plurality of transducer elements, a first transmit surface, a first matching layer and a first backing layer and the second transducer stack comprises a second plurality of transducer elements, a second transmit surface, a second matching layer and a second backing layer wherein each the first transducer stack and the second transducer stack are longitudinally adjacent to each other and the first transmit surface and second transmit surface are separate, and a circuit is connected to both the first plurality of transducer elements and the second plurality of transducer elements; wherein the circuit is capable of activating a number of elements equal to a total of the first plurality of transducer elements and the second plurality of transducer elements and wherein such activation is mutually exclusive.

An acoustic lens material may cover both the first transducer stack (e.g., in some embodiments, including the first transmit surface of the first transducer stack) and the second transducer stack (e.g., in some embodiments, including the second transmit surface of the second transducer stack). In addition, or in the alternative, as described further below, such acoustic lens material may fill the colinear space between first transducer stack and the second transducer stack. For greater clarity, although an acoustic lens material may unitarily and jointly cover the outermost surface of the first and second matching layers (e.g., radiation regions of the first transmit surface and the second transmit surface), the first transmit surface of the first array (for example, for generating high frequency waves) and the second transmit surface of the second array (for example, for generating low frequency waves) are separate, there is no overlap therebetween and as such there is no common radiation surface.

The acoustic lens (also referred to as acoustic lens material) is used to focus an ultrasound beam at the target distance and usually protects each of the first and the second matching layers as a medical ultrasound transducer without an acoustic lens could directly contact the target. As such, the matching layers (also referred to as acoustic matching layers), as described further below, are used to propagate as much ultrasound energy as possible, by matching the target medium with a piezoelectric material (also referred to herein interchangeably as the first and second arrays and the first and second plurality of transducer elements), such piezoelectric material being employed to convert electrical energy into ultrasound energy and vice versa. In the present embodiments, the respective matching layers assist in transferring the ultrasound energy from the first and second plurality of transducer elements to the medium (target tissues). Without the matching layers, the large impedance difference between the acoustic source (about 33 Mrayls) and the target (about 1.5 Mrayls) would result in loss of transmission and receipt of acoustic energy of up to 90 percent at the interface between the source and the target.

With the present invention, the first and second matching layers are located between the respective first and second plurality of transducer elements and a unitary acoustic lens. Such matching layers comprise materials that are conducive to achieving better energy transfer, such as, for example, epoxy, polyurethane, polystyrene, etc. The first and second backing layers, as described further below, may be used to absorb the ultrasound energy towards the system. The backing layers prevent the backward emitted sound waves to echo and ring back into the transducer for detection. There are a variety of configurations known in the art for selecting backing materials (materials, thickness, adhesive means) and applying the same to the back of transducer elements.

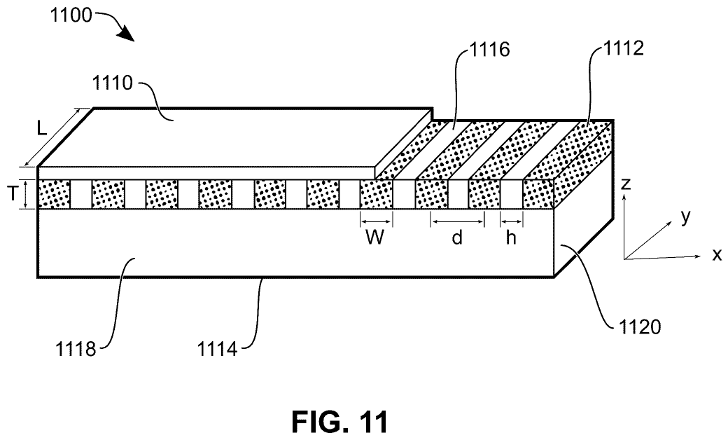

Geometry of each of the first and second piezoelectric transducer arrays of the present invention are generally composed of N piezoelectric elements having a thickness T, a width W and a length L, spaced by a distance d=W+h (d corresponds to each array's pitch and h is a filling material's width) and organized as shown by way of example in FIG. 11, described further below. Within each transducer array, the piezoelectric elements are polarized in the thickness direction (z-axis) and are bonded to each other by, for example, a non-conductive resin. Generally, the thickness T depends on the desired operating frequency of each array, which is approximately equal to a half of the wavelength in the piezoelectric material. As is known in the art, to avoid the parasitic grating lobes, the width W is designed to respect the Nyquist criterion. Within an embodiment of the present invention, the pitch (d) of both the first plurality of transducer elements and the second plurality of transducer elements is either the same or is substantially the same, regardless of each array's generated frequency range.

In one embodiment of the present invention, the thickness of the first plurality of transducer elements producing a higher frequency bandwidth (e.g., the first piezoelectric layer) is lower than the thickness of the second plurality of transducer elements producing a lower frequency bandwidth (e.g., the second piezoelectric layer) due to the differential in frequencies therebetween. For this reason, production and manufacturing of each of the first transducer stack and the second transducer stack would reflect such differences. For example, cuts between elements or in the composite of the thicker second piezoelectric layer would require a thicker saw blade than for the cuts in the thinner first piezoelectric layer.

In some embodiments, the first array is linear and produces a high frequency bandwidth and the second array is a phased-array and produces a relatively lower frequency bandwidth. The general arrangement of the two arrays within the ultrasound probe of the invention may provide that each is substantially flat and that a length of the first array is greater than a length of the second array and that a width of the first array is less than a width of the second array. In this way, while the first array is longitudinally aligned with the second array, there is a dummy gap defined by the extended length of the first array (for example linear array) as compared to the second array (for example, phased array). Such gaps are best illustrated with reference numeral 322 in FIG. 3 of the present description.

In some embodiments of the present invention, a dual array probe is used to image at different depth ranges for optimized selection of frequency for different image depths. In this case, a first array at an imaging end of the probe may be used to image at shallower depths for improved axial resolution and focus at these depths. This first array may, for example, be a linear array operating at 5-15 MHz. A second array at the same imaging end of the probe as the first array may be used to image at deeper depths with improved penetration and correspondingly deeper focus. The second array may, for example, be a phased array operating at 1-5 MHz. Since a probe as provided in the present invention may perform ultrasound imaging using either of these arrays, it may be considered as having a wide frequency range that spans 1-15 Mhz. This dual array probe may be particularly desirable in a portable form, especially although not exclusively for emergency use, because the number of probes required to be carried around would be reduced. By separating the two transducer stacks, the focal depths of each of the first array (e.g., operating at a high frequency) and the second array (e.g., operating at a lower frequency) can be customized to each array. In some embodiments, the second transmit surface may have a larger relative surface area as compared to the surface area of the first transmit surface, generating a first array of a higher relative frequency. Within the scope of the present invention, the pitch of the first array and the pitch of the second array may be either the same or substantially the same. The present invention thus presents a general solution for a dual array probe with a combined high frequency array/low frequency array which allows the apertures, frequencies and foci to be electronically selectable for optimal measurements in two different modes. The two different modes may be changeable in a variety of ways: e.g., via depth input by a user (e.g., higher depths result in imaging using the second array whereas shallower depths result in imaging using the linear array), automatically through AI-determined pre-set selection, depending on the clinical application, and/or manually by the operator (for example by way of button(s) on the probe itself or by display GUIs, whether through tactile interaction with such display or by audio control/commands, to optimize image quality.

In an example embodiment, a first array/first transducer stack is linear, and a second array/second transducer stack is a phased array (sector array). Generally, phased array transducers known in the art are typically 2-3 cm long, consisting of 64-128 elements and provide a sector field of view by all of the elements firing to create a single waveform. Small delays in element firing allow for electronic field steering and focusing without moving the ultrasound probe. All elements will be fired multiple times with different degrees of steering to create an ultrasound image. Echoes are detected by all elements and entered into an algorithm to form the image and lateral resolution decreases at the bottom of the image due to increasing lateral separation of adjacent lines at higher depths. The sensitivity of the ultrasound image reduces at extremes of steering (e.g., when ultrasound beams are steered to the leftmost and rightmost parts of the sector image) and lateral resolution is best in the center of the field of view due to a larger effective aperture. The benefits of a phased array include a small-faced transducer allowing for imaging in small spaces and being able to change the focus of the ultrasound beam. High elevational (azimuthal) resolution throughout the field of view is assured by a focused transmit beam and by sweeping the focus of the receiver is synchrony with the range of returning echoes. In an example embodiment, elevational (azimuthal) resolution may vary from 2 to 5 mm throughout the field of view while range resolution may be 1.5 mm. Although this type of imaging system has proven particularly useful for the delineation of left ventricular spatial geometry by the identification of endocardium, myocardium, papillary muscles and interventricular septum, it may be generally used in a wide variety of the abdomen, cardiac, lung and vascular applications.

Generally, when imaging, a linear array/linear transducer comprises multiple adjacent elements of the array combining to produce an ultrasound beam that is emitted at 90 degrees to a transducer head. These multiple elements work together in order to achieve a wider aperture and highly useful beam shape. Successive groups of adjacent elements are pulsed sequentially along the scan head, and each pulsed group produces a scan line that together, make up the many scanlines of an ultrasound image. In this way, only parallel ultrasound waves are emitted, allowing for the same lateral resolution in the near-field and the far-field, and resulting in an image with a rectangular field of view. Linear arrays are used, for example, for small parts, superficial parts, vascular (including vessels like carotids and jugular vein), breast, thyroid, lungs, musculoskeletal imaging (MSK), and ultrasound-guided procedures like placing a central line, a nerve block in regional anesthesia, or other procedure where visualizing a needle may be desirable. A linear array provides a suitable view for those working in intensive care, emergency medicine, and anesthesiology.

As such, in a broad aspect of the present disclosure, there is provided a wide frequency range, dual array ultrasound imaging probe that has two different operational modes which permit using the same ultrasound probe for multiple purposes and multiple scanning options. In one embodiment, the same transducer may be used, for example, in both a high frequency imaging mode and a low frequency imaging mode without the requirement to switch/remove probe heads or entirely change from one probe type to another.

In an example embodiment, the probe described herein provides that the first array comprises a first plurality of transducer elements disposed on the imaging end (e.g., the first piezoelectric elements) which operate in a high frequency band (e.g., between 5 to 11 MHz). The second array may have a second plurality of transducer elements disposed on the imaging end (e.g., second piezoelectric elements) operate in a low frequency band (e.g., between 1 to 5 MHz).

An advantage of the architecture of one embodiment of the dual array probe of the present invention is as follows. The provision of a first array being linear and the second array being a phased array and wherein each is non-curved, creates a highly versatile wide frequency probe for optimal vascular scanning, optimal cardiac scanning and acceptable abdominal scanning. Some traditional wide frequency probes pair a curved low frequency array with an adjacent high frequency array that also has a curved footprint. Such a configuration has several drawbacks. First, high frequency arrays are typically used for more superficial imaging (e.g., vascular). However, due to the curved configuration of the high frequency array, in order to maintain full scanning surface contact, there would necessarily be distortion and compression of vascular structures. This makes their value in scanning superficial structures questionable. Second, because the low frequency array is natively curved, it is typically configured to have a transducer element configuration that is optimized for curvilinear imaging. For example, the curvilinear array may have a pitch that is higher than what would be used for a linear array or a phased array. If attempts were made to perform phased array imaging using the curved array, this would result in grating lobes on the outer edge of the image which, in turn would result in a narrower field of view (FOV). This would be in addition to the drawback of the sub-optimal footprint of the curved array for cardiac imaging.

In contrast, the present embodiments position a high-frequency linear stack beside a phased array stack, with the pitch for both being configured for typical linear and phased array stacks. This has a two-fold advantage: (i) no grating lobes in the phased array images, which in turn allows for a wider FOV, and (ii) no compressibility during vascular imaging when using the linear stack. The lack of a pure curved array for abdominal imaging may generally be found to be acceptable because in many emergency departments, phased array probes are used as their primary probe of choice because it obtains optimal cardiac imaging (e.g., better than a traditional curvilinear probe) while still being able to acquire acceptable abdominal images.

Accordingly, the selection of a piezoelectric element pitch (“d” in FIG. 11) which is the same or substantially similar between each of the linear array and the phase array is functionally purposeful and not only provides manufacturing efficiencies but maintains a standard FOV as would be expected from a conventional phased array. Wherein transducer elements emit an ultrasound wave with a wavelength λ, it is preferred that a pitch of the second array of the present invention (the phased array) be less than or about equal to λ/2 (½ frequency of a wavelength or “half wavelength rule”), in order to avoid grading lope artifacts.

A key value proposition of one embodiment of the invention, wherein in a dual array probe a first array is linear and a second array is a phased array, first and second transmit surfaces being substantially non-curved, and wherein the preferred selection of a piezoelectric element pitch is the same or substantially similar between each of the linear array and the phase array, is a product which practically and functionally balances the competing operational requirements of each array. While maintaining a generally accepted pitch for a linear array, the lateral resolution of the resultant images produced by the probe remains unchanged and as expected. By only increasing the pitch of the phased array a modest amount (but still within the half-wavelength rule so as to avoid grading lobe artifacts) as compared to conventional phased arrays, the FOV of the resultant images produced by the probe remains as expected. This balance results in tangible and practical benefits, particularly in emergency practise and settings. With the dual array probe of the present invention, there is no sacrifice of cardiac imaging quality in order to provide higher abdominal scanning quality. Instead, the probe of the present invention offers, as a primary focus, excellent vascular and cardiac scanning quality, while providing sufficient abdominal scanning using the phased-array (second transducer) in order to identify issues as required in an emergency (quick scan and review) setting.

The components are discussed in this section are to provide a functional understanding of the system of the present invention. It is to be understood that typically, ultrasound systems focus sound waves along a given scan line so that the waves constructively add together at a desired focal point. As the sound waves propagate towards the focal point, they reflect off on any object they encounter along their propagation path. Once all the reflected waves have been measured with the transducers, new sound waves are transmitted towards a new focal point along the given scan line. Once all the sound waves along the given scan line have been measured, the ultrasound system focuses along a new scan line until all of the scan lines in the desired region of interest have been measured. To focus the sound waves toward a particular focal point, a set of transducer elements are energized with a set of time-delayed pulses to produce a set of sound waves that propagate through the region of interest, which is typically the desired organ and the surrounding tissue. This process of using multiple sound waves to steer and focus a beam of sound is commonly referred to as beamforming. Once the transducers have generated their respective sound waves, they become sensors that detect any reflected sound waves that are created when the transmitted sound waves encounter a change in tissue density within the region of interest. By properly time delaying the pulses to each active transducer, the resulting time-delayed sound waves meet at the desired focal point that resides at a pre-computed depth along a known scan line. The amplitude of the reflected sound waves forms the basis for the ultrasound image at this focal point location. Envelope detection is used to detect the peaks in the received signal and then log compression is used to reduce the dynamic range of the received signals for efficient display. Once all of the amplitudes for all of the focal points have been detected, they can be displayed for analysis by an ultrasound user/operator. Since the polar coordinate system in which the ultrasound system usually operates does not match the display cartesian coordinate systems, a coordinate transformation called scan conversion needs to be performed before being displayed on a display screen (for example on a multi-purpose electronic device) or on a monitor.

A beamformer control unit is responsible for synchronizing the generation of the sound waves and the reflected wave measurements. A controller inputs/outputs commands on the region of interest in terms of width and depth. This region gets translated into a desired number of scan lines and a desired number of focal points per scan line. The beamformer controller begins with the first scan line and excites an array of piezo-electric transducers with a sequence of high voltage pulses via transmit amplifiers. Typical performance numbers for the amplifiers is up to ±100 V and up to ±2 Amps for each piezo-electric element. The pulses go through a transmit/receive (Tx/Rx) switch, which prevents the high voltage pulses from damaging the receive electronics. These high voltage pulses are properly time delayed so that the resulting sound waves can be focused along the desired scan line to produce a narrowly focused beam at the desired focal point. The beamformer controller determines which transducer elements to energize at a given time and the proper time delay value for each element to properly steer the sound waves towards the desired focal point. As the sound waves propagate toward the desired focal point, they migrate through materials with different densities. With each change in density, the sound wave has a slight change in direction and produces a reflected sound wave. Some of the reflected sound waves propagate back to the transducer and form the input to the piezo-electric elements in the transducer. The resulting low voltage signals are scaled using a variable controlled amplifier (VCA) before being sampled by analog-to-digital converters (ADC). The VCA is configured so that the gain profile being applied to the received signal is a function of the sample time since the signal strength decreases with time (e.g., it has traveled through more tissue). The number of VCA and ADC combinations determines the number of active channels used for beamforming. It is usual to run the ADC sampling rate a multiple higher (for example, 4 times or higher) than the transducer center frequency. Once the received signals reach the Rx beamformer, the signals are scaled and appropriately delayed permitting a coherent summation of the signals. This new signal represents the beamformed signal for one or more focal points along a particular specific scan line. The beamformer operations are typically performed in application-specific integrated circuit (ASIC), field-programmable gate array (FPGA), DSP or a combination of these components. The number of channels used in beamforming determines the input/output (I/O) requirement as well as the processing requirement to perform digital beamforming.

Once the data is beamformed, depending on the imaging modes, various additional processing may be carried out. For example, it is common to run the beamformed data through various filtering operation to reduce out band noise. In B (Brightness) mode, demodulation followed by envelope detection and log compression is the most common practice. Several 2D noise reduction and image enhancement functions may also be performed in this mode followed by scan conversion to fit a desired display system (for example, a display screen on a multi-purpose electronic device). These may be implemented in FPGA, ASIC, DSP or any combination of these components.

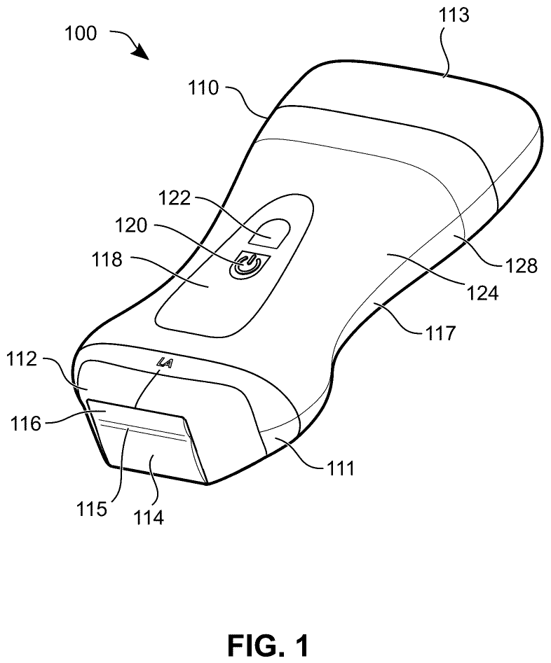

Referring to FIG. 1, shown there generally as 100 is a perspective view of a wide frequency/dual array ultrasound probe, in accordance with at least one aspect of the present invention. As illustrated, the dual array ultrasound probe comprises a body 110, a head 111, an imaging end 112 and a non-imaging end 113 (disposed on the body opposite to imaging end 112). A centre of the body 117 may be curved inwards forming a narrower portion of the body, as compared to imaging end 112 and non-imaging end 113, for ease in grip and handling of the probe by an operator. In some embodiments, a control panel 118 may be situated on a front face 124, including buttons 120 and/or 122 which may be on/off controls and/or multi-function buttons (for example, to switch between arrays), as required. In other embodiments, the probe body may be free of buttons, with operations being controlled via a communicable connection to a multi-purpose electronic device, which by wired connection or wirelessly provides inputs and commands to the probe. Dual array probe 100, at imaging end 112, is defined in part by a unitary acoustic lens covering a first section/transducer head 116, under which is oriented a first transducer stack (comprising for example, a first matching layer, a first array/first piezoelectric array and a first backing material shown best in FIG. 6, a second section/transducer head 114, under which is oriented a second transducer stack (comprising for example, a second matching layer, a second array/first piezoelectric array and a second backing material also shown best in FIG. 6, and dividing/demarcation line 115 therebetween, and under which may be generally disposed colinear space (as shown in FIG. 6.

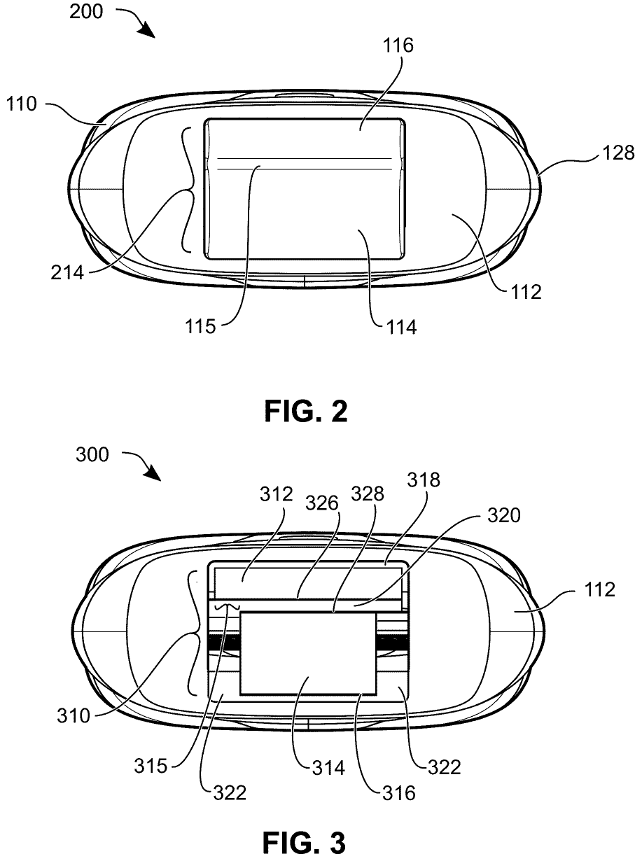

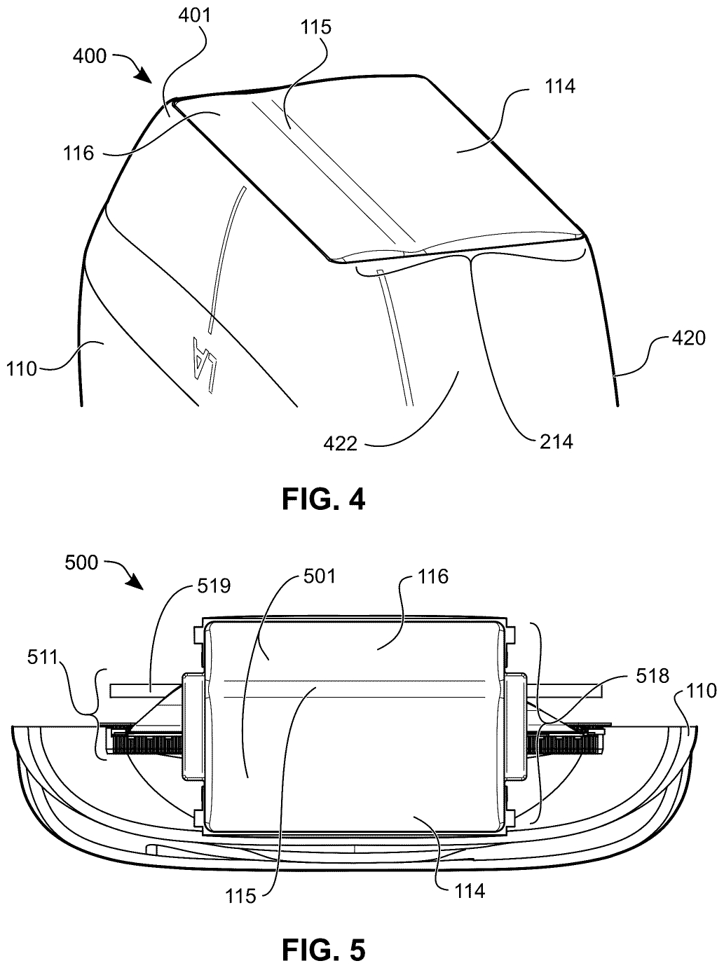

In FIG. 2 there is shown generally at 200, a top plan view of a dual array probe in accordance with at least one aspect of the present invention, such view providing an enhanced view of probe body 110 with imaging end 112 comprising a unitary acoustic lens 214 covering a first section/transducer head 116, a second section/transducer head 114 and dividing/demarcation line 115. Further, FIG. 4, at 400 illustrates a partial cut-away elevation perspective view of features and orientation of the imaging end of ultrasound probe 110 in accordance with at least one aspect of the present invention, such view showing probe body 110 and imaging end (also interchangeably referred to as end cap 401) comprising a unitary acoustic lens (entire region 214) which is covering a first section/transducer head 116, a second section/transducer head 114 and dividing or demarcation line 115.

FIGS. 3 and 5 complementarily show a top plan view of a dual array probe of the present invention. FIG. 3 shows a partial cut-away view, whereas FIG. 5 shows acoustic lens material 501 covering the dual array/transducer stacks. In FIG. 3, such dual array/transducer stacks are exposed and not covered by acoustic lens material thereby exposing the first matching layer (of the first transducer stack) and the second matching layer (of the second transducer stack). For greater clarity and as described further below, FIGS. 3 and 5 provide two different levels of cut-away from the top plan view of FIG. 2, with FIG. 2 showing a fully intact imaging end, FIG. 5 having all or part of end cap 401 removed but the unitary acoustic lens material intact and visible, and FIG. 3 having both end cap 401 and unitary acoustic lens material removed thereby most clearly illustrating the size, relative positioning and orientation of the first transducer stack and the second transducer stack.

In an embodiment of the invention, the first section/transducer head 116 (as shown in FIG. 1) comprises a first transducer stack (shown as 612 in FIG. 6) which in turn comprises a first array/first piezoelectric array (shown as 626 in FIG. 6) which is preferably a high frequency array, e.g., linear, and the second section/transducer head 114 (as shown in FIG. 1) comprises second transducer stack (shown in sectional cut-away as 614 in FIG. 6) which in turn comprises a second array/second piezoelectric array (shown as 628 in FIG. 6) which is preferably a low frequency array, e.g., a phased array. A mode of operation may enable switching from an activation of one array to the activation of the other array wherein only one of the two arrays is activated at a time (e.g., mutually exclusive activation).

The second transducer stack, e.g., being a phased array, may comprise a transducer head with a smaller relative length footprint than the relative length footprint of the first transducer stack, e.g., a linear array. This is shown best in FIG. 3, wherein a top plan view of a dual array probe is shown as 300, comprising imaging end 112 having a linear first transmit surface 312 (top surface of first matching layer, under which is the remainder of linear first transducer stack 612, as shown best in FIG. 6) and a phased-array second transmit surface 314 (top surface of second matching layer, under which is the remainder of phased array second transducer stack 614, as shown best in FIG. 6). Together the two stacks are shown as 310. A distance 315 represents a differential between a length of the linear matching layer and a length of the phased array matching layer, such differential creating two (right side and left side) gap areas 322 lateral to the second transducer stack. Gap areas 322 (also called dummy gaps) are described further below. Further, such length differential 315 is created by linear first transducer stack 612 comprising a greater relative number of piezoelectric elements in first array 626 (thereby having a longer footprint) as compared to the number of piezoelectric elements in second array 628 in second transducer stack 614. As such, the dummy gap is a function of the relative number of piezoelectric elements in each array. Within the scope of the invention, a unitary acoustic lens covers both the first transducer (first array) and the second transducer (second array) and as such, there is a functionality trade-off in selecting the total number of total elements and the number of elements activatable in each array, particularly to enable a phased array footprint that, with the dummy gaps, remains useful for cardiac imaging.

In FIG. 5 there is shown generally as 500 a top sectional plan view of the dual array probe as provided in FIG. 3, but with a unitary acoustic lens 501 covering each of: i) a first section/transducer head 116 (e.g., the linear transducer head); ii) a second section/transducer head 114; and iii) and dividing/demarcation line 115 therebetween (where 116, 114 and 115 are collectively surfaces 518). Body 110 houses first (linear) transducer stack (seen as 318 as seen in FIGS. 3 and 612 in FIG. 6) and second (phased array) transducer stack (seen as 316 in FIGS. 3 and 614 in FIG. 6A) along with below, electronic components such as transducer connectors, flexible PBCs, circuits and switches (shown for reference as components 511 and 519).

FIG. 6 shows a side view in partial cut-away of a dual array ultrasound probe, in accordance with at least one aspect of the present invention, exposing both a high frequency (e.g., linear) transducer stack 612 and a low frequency (e.g., phased-array) transducer stack 614, including flexible PCB and other electronic architecture under each transducer array. A description of these components will refer to FIG. 6 and FIG. 11, a schematic diagram of a geometry of a transducer stack with dimensional guide reference. FIG. 6 illustrates generally at 600 a set of two transducer stacks 612 and 614 which are longitudinally aligned and separated by a colinear space 623, such space, after manufacturing, being filled with acoustic lens material, shown as 621. Generally, each transducer stack is a substantially a rectangular block/rectangular prism as illustrated in FIG. 11, comprising six faces: a first, a second and two lateral (also referred to as surfaces), where all the faces of the prism are rectangles such that all the pairs of the opposite faces are congruent. As such each transducer stack comprises a first surface (shown by way of example as matching layer 1110 in FIG. 11) extending along, adjacent, and parallel to an imaging end of a probe (when in situ, with the probe) such surfaces, for example, being under first transmit surface 312 and second transmit surface 314, shown in FIG. 3. Each transducer further comprises a first lateral surface (shown as 1118 in FIG. 11) extending perpendicular to its respective first surface. A first lateral surface of the first transducer stack (shown as 326 in FIGS. 3 and 6) faces an opposite first lateral surface of the second transducer stack (shown as 328 in FIGS. 3 and 6). Each transducer stack further comprises a second lateral surface shown as 1120 in FIG. 11. In this way, and by this orientation, imaging end 112 of dual array probe 100 comprises a first array and a second array that are in substantially the same plane, parallel to an imaging contact surface. Also, when each of the two transducer stacks are in place within the probe first lateral surface of the first transducer stack 326 faces towards first lateral surface of the second transducer stack 328, separated by colinear space 623.

A dual array probe of the invention generally comprises a body (110 in FIG. 1) comprising an imaging end (112 in FIG. 1), a non-imaging end (113 in FIG. 1), a front face (124 in FIG. 1), a back face (420 in FIG. 4), a first side (422 in FIG. 4) and a second side (128 in FIG. 1). To show both transducer stacks in FIG. 6, this view is depicted as such components would be, in place, from a second probe side (from 128). If one were to view a sectional cut-away from either the front face 124 or the back face 420, due to orientation of the transducer stacks, one of the two transducer stacks would be in the foreground. For example, in a view from the front face 124, the first transducer stack would be in the foreground. And in a view from the back face 420, the second transducer stack would be in the foreground. Other arrangements of PCB connection to the transducer stacks are contemplated as being within the scope of the present invention.

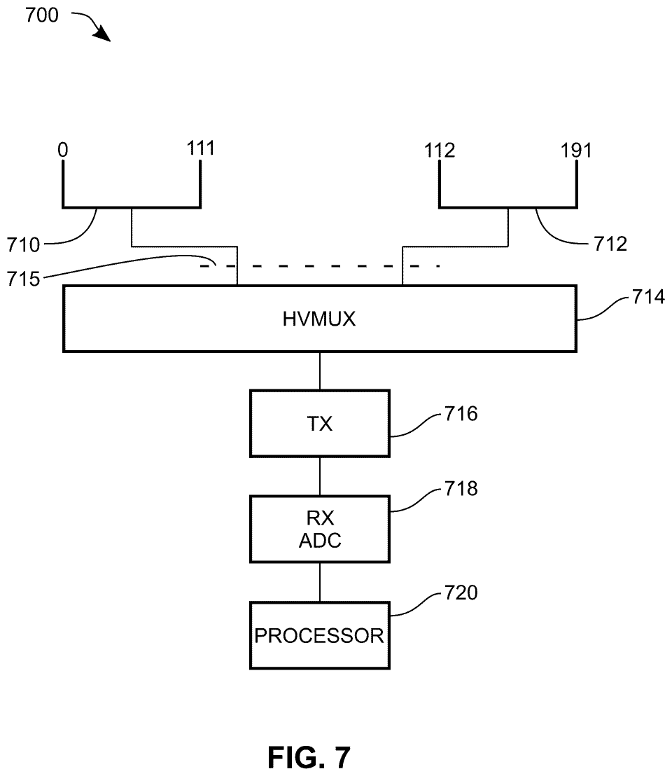

First transducer stack 612 may be a linear array, the elements of such stack being, at least, first matching layer 622, first piezoelectric array 626 and first backing layer 630. Second transducer stack 614 may be a phased array, the elements of such stack being, at least, a second matching layer 624, second piezoelectric array 628 and second backing layer 633. An acoustic lens material 620 unitarily cover a plurality of surfaces of both the first transducer stack and the second transducer stack, as illustrated. In addition, acoustic lens material 621 fills colinear space 623. Electronic circuitry connected to each transducer stack is shown as first flexible PCB strip 634, and second flexible PCB strip 636 extending to interposer FPCBA 640, interposer FPCBA connector (transducer side) 638 and interposer FPCBA connector (system side) 642. High voltage MUX (HVMUX) is provisioned in the main PCB and shown in FIGS. 7 and 10.

In this example dual transducer stack assembly, as shown, second flexible PCB strip 636 wraps around a perimeter of at least three sides of backing material 633, it being bonded (for example, glued) directly to electrodes of the piezoelectric ceramic elements 628 (for example, electrical connections made between individual traces of flexible PCB strip and the positive and ground electrodes in locations of each intended transducer element. For instance, wires can be soldered onto the positive and ground electrodes and attached to individual traces of the flexible circuit. In contrast, first flexible PCB strip 634 extends along a portion of first lateral surface 326 on first transducer stack 612 and is not directly affixed or bonded to piezoelectric ceramic elements 626, instead connected by a wire assembly thereto. First flexible PCB strip 634 and second flexible PCB strip 636 meet at interposer FPCBA connector (transducer side) 638.

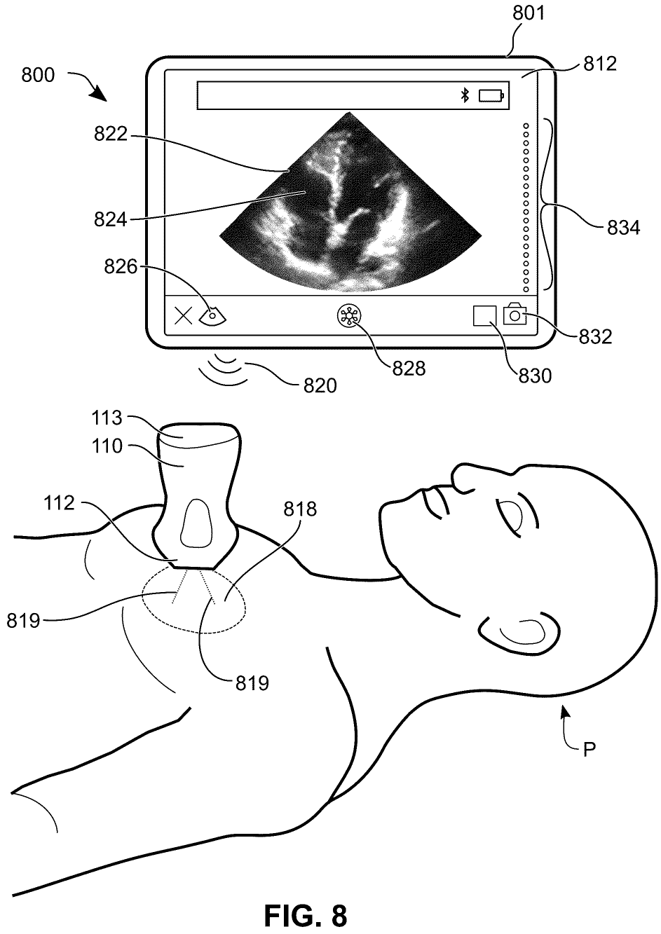

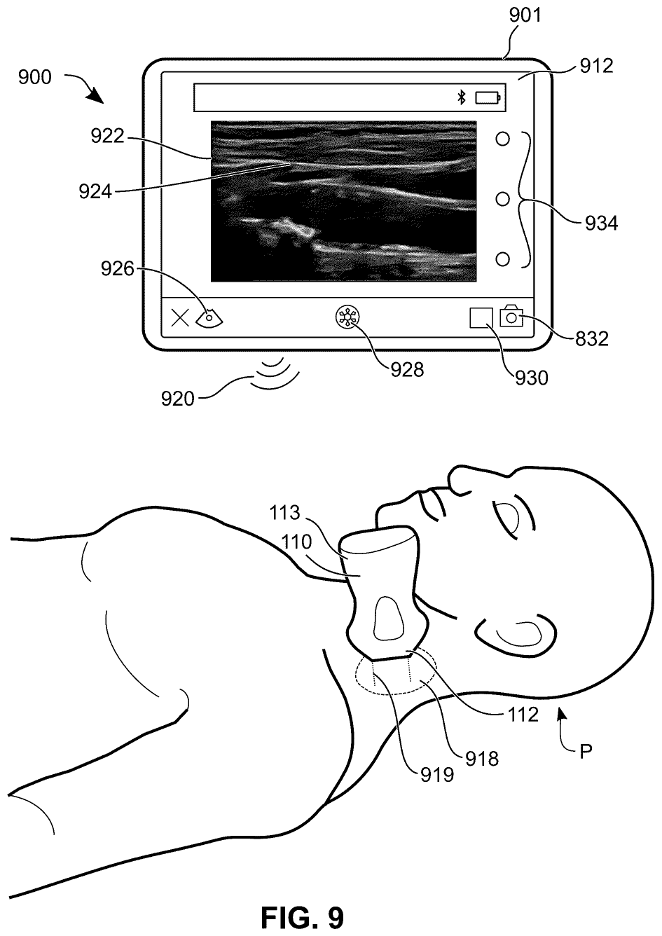

In an example embodiment where the first transducer stack is a linear array and second transducer stack is a phased array, the second piezoelectric array 628 may have fewer elements than the first array 626, e.g., a relatively smaller number of transducer elements positioned therein as compared to the number of transducer elements (in first array 626) positioned in linear transducer stack 612. Due to a comparatively more limited number of transducer elements, ultrasound signals (shown as 819 in FIG. 8) therefrom can be projected in a phased manner in multiple directions to obtain a broader field of view. This results in a fan- shape sector image 822 (as shown in FIG. 8) being generated. In contrast, a linear-array transducer has a transducer head with a larger footprint (lengthwise) on which a comparatively larger number of transducer elements are positioned in a line. Using the larger number of transducer elements, beamforming is performed on successive groups of adjacent transducer elements to direct ultrasound signals into a volume of tissue being imaged. The ultrasound signals are projected in a single direction orthogonal to the surface of a transducer head and each projected ultrasound signal (919 in FIG. 9) forms a scanline, so that the cumulative scanlines form a rectangular ultrasound image 922 (as shown in FIG. 9). As a phased-array transducer directs ultrasound signals 819 in multiple directions from a smaller number of transducer elements, a distance between each scanline increases as the distance from the transducer head increases. This may reduce the lateral resolution in a far field of the sector image generated (e.g., the ability to resolve between two adjacent objects in a direction perpendicular to the direction of beam travel, but in-plane with the ultrasound imaging being generated). In contrast, since the linear-array transducer projects parallel ultrasound signals 919 from multiple locations on the transducer head, there is a consistent distance between adjacent ultrasound signals in both the near field and the far field of the generated rectangular image 922. As a result, images generated using the linear-array transducer may not suffer from a degradation in lateral resolution as imaging depth increases.

| | High Frequency | | Low Frequency | | |
|---|---|---|---|---|---|
| Parameter | Array (614) | | Array (612) | | Overall |
| Frequency | 5-15 | | 1-5 | | |
| Range (MHz) | | | | | |
| Pitch | 0.26 (260) | | 0.26 (260) | | |
| (mm/microns) | | | | | |
| FOV/active | 29.19 × 4.25 | mm | 20.8 × 11 | mm | |
| dimension | | | | | |
| Lens dimension | 32.16 × 7 | mm | 32.16 × 15.55 | mm | 32.16 × 22.55 mm |
| Elevation Focus | 30 | mm | 80 | mm | |

| | | | |
|---|---|---|---|
| Array Arrangement | Linear | Linear/Phased | |
| # Elements | 112-128 (e.g., 112) | 64-80 (e.g., 80) | Example total |
| | | | elements: 192 |
| Connections | Connect to | Connect to | Each element |
| | S0-S111 pins | S112-S191 pins | connections are |
| | | | independent with |
| | | | common GND |
| Axial Resolution | Very Good | Average | |
| Far Field | Good | Adequate | |
| Lateral Resolution | | | |
| Medical | Vascular, | Cardiac, Lung, | |
| Applications | Superficial | Abdominal | |

In employing the switchable dual array probe of the present invention, the phased-array transducer stack 614 may be used to image, for example, the heart or lungs because even though there is relatively poorer far field lateral resolution, its small transducer head footprint results in phased multi-directional ultrasound signal 819 projection. When this array is activated, e.g., selected by input to the wide frequency probe, only the second array of piezo elements are activated by a controller and only the phased array signals are generated at the imaging end 112 of the probe. The first array of piezo elements remains inactive. Due to the footprint of the phased array, when activated, the ultrasound signals 819 are projected in a variety of different directions through for example, the space between, for example, a patient's ribs, so as to allow for imaging of organs such as the heart, thereunder, as shown in FIG. 8. By an input to the dual array probe, an operator using the dual array probe of the invention may simply and easily switch arrays as required without the need to switch between different ultrasound probes or switch ends of a probe, during an examination, which steps may be inefficient, messy and cumbersome. If a switch is made between activation of the second transducer stack and second piezo array (for example a phased array) to, instead, activation of the first transducer stack and first piezo array (for example a linear array of high frequency to provide better axial resolution in the near field) only the first array of piezo elements is activated by a controller and only linear array signals are generated at the imaging end of the probe.

FIG. 11 illustrates at 1100 a geometry of a piezoelectric transducer (comprising at least matching layer 1110, piezoceramic elements 1112, filling material 1116 and backing material 1114) as may be employed within the present invention. Both linear and phased transducer arrays are composed of N piezoelectric elements 1112 having a thickness T, a width W and a length L, spaced by a distance d=W+h (d corresponds to each array's pitch and h is the filling material 1116 width) and aligned as illustrated in FIG. 11. The elements are polarized in the thickness direction (z-axis) and are bonded to each other by, for example, a non-conductive resin. The thickness T depends on the desired operating frequency, which is, as described above, approximately equal to a half of the wavelength generated in the piezoelectric material. To avoid parasitic grating lobes, the width W must respect the Nyquist criterion at the desired operating frequency. Within one aspect of the present invention, thickness T of the phased array element array is thicker than the thickness T of a linear array. This is illustrated in FIG. 6 (626 being measurably thinner than 628). Further, within the present invention, the transducer element array of a phased-array transducer may be configured to have a pitch which is the same or substantially the same as the pitch of the transducer element array of a linear array transducer and it is preferably between 250 and 260 microns, and in an example embodiment, 260 microns. There may be other acceptable pitches. For example, in some embodiments, different pitches shared by both the linear array and phased array, for use in the dual array probe of the invention, can be used as long as the array centre frequency (operational frequency) of the phased array meets the Nyquist criterion for mitigating grating lobes, as discussed herein.

In an example embodiment, the number of piezoceramic elements 1112 is greater in the first array/linear array as compared to the number of piezoceramic elements 1112 in the second array/phased array. Due to these geometry preferences which are designed for optimal functionality of each respective array, the length of the first array is greater than the length of the second array, and the width of the first array is less than the width of the second array and, as can be seen best in FIG. 3, and a gap or dummy gap 322 is thereby created when the two transducer stacks are linearly aligned. This gap may be filled with acoustic lens material forming a uniform rectangular block comprising both dual stacks not only for cosmetic reasons and acoustic optimization, but for ease in manufacturing.

Referring to FIG. 7, circuitry controls the activation of a total number of transducer elements (a first plurality of transducer elements 710 plus a second plurality of transducer elements 712), wherein the first plurality of transducer elements 710 has a larger number of transducer elements than the second plurality of transducer elements 712 and wherein such activation is mutually exclusive. For greater clarity, mutually exclusive means that when a high voltage multiplexer circuit (HVMUX 714) receives an input via processor 720 to activate transducer elements, it can only activate one subset of the total number of transducer elements, at any one time (subset 710 or subset 712) and not both. The circuit (for example, the HVMUX) receives commands from a processor (such as at 720 in FIGS. 7 and 1030 in FIG. 10) and activates the first plurality of transducer elements 710 starting from a start element which is either 0 or 1 and, mutually exclusively, and which activates the second plurality of transducer elements 712 starting from a start element which is neither 0 nor 1. For orientation, above dashed line 715 represents the components shown, in greater detail, in FIG. 6.

In one aspect of the invention, the first plurality of transducer elements 710 is part of a high frequency first transducer stack, which may be a linear transducer stack that includes 112 piezoelectric elements (each element being addressed using an addressing scheme from either 0-111 or 1-112). The second plurality of transducer elements 712 may be part of a low frequency second transducer stack, which may be a phased array transducer stack and may include 80 piezoelectric elements (each element being addressed using an addressing scheme from either 112-191 or 111-192). Notably, in this configuration, the element addressing scheme for the second plurality of transducer elements 712 continues on from where the addressing scheme of the first plurality of transducer elements 710 ended; so that when the first and second plurality of transducer elements 710, 712 are taken together, the connections to the HVMUX 714 may consider them as addressing a contiguous block of transducer elements addressed from 0-191. This addressing scheme simplifies electrical connection to the HVMUX 714 so that the same set of electronics that connect to standard single arrays having 192 elements can be re-used to connect to multiple arrays without modifying the underlying electronics. To support connection of the same electronics to the dual array configuration of the present embodiments, changes need only be made to the firmware/software to activate the first linear array transducer independently of the second phased array transducer.

HVMUX 714 provides a number of fixed aperture configurations that can be selected by specifying an aperture number in the TX (Transmit 716) or RX (Receive 718) structures. HVMUX 714 in one aspect may comprise an embedded microcontroller (MCU) to receive external commands and set analog switch states, integrated circuits (ICs), and power sequencing switch. For example, HVMUX 714 may be connected to three 64-signal arrays containing a plurality of high voltage analog switches. A variety of HVMUX suitable for use within the invention are commercially available including, but not limited to, those available from Microchip Technology, Inc., STMicroelectronics International N.V, Texas Instruments Incorporated, Hitachi Power Semiconductor Device, Ltd. and Monolithic Power Systems, Inc.

An example operation of the dual array ultrasound probe of the present invention is illustrated in FIGS. 8 and 9. An ultrasound examination of patient P is shown generally at 800, wherein a dual array ultrasound probe 110 comprising imaging end 112 and non-imaging end 113 is under operation and is scanning a chest region 818 of patient P. At imaging end 112 is provisioned a unitary lens surface (as described fully herein) and thereunder are provisioned two separated but longitudinally aligned transducer stacks. In the examination depicted in FIG. 8, the phased array transducer stack is operational and only the subset of the total number of transducer elements dedicated to such an array are activated, and only the second transmit surface is conveying signals/beams, 819. For greater clarity, while the entirety of the imaging end 112 may be depressed against patient P during this examination, only the second transmit surface is conveying signals/beams. Dual array ultrasound probe 110 may be wirelessly connected at 820 to interface screen 812 of multi-purpose electronic device 801 wherein an image 822 of heart 824 may be displayed thereon. Interface screen 812 may comprise controls and guides including but not limited to: mode selector 826, freeze button 828, video icon 830, screen capture icon 832 and depth indicator 834.

In FIG. 9, ultrasound examination of patient P is shown generally at 900, wherein dual array ultrasound probe 110 comprising imaging end 112 and non-imaging end 113 is under operation and is scanning a neck region 918 of patient P. At imaging end 112 is provisioned a unitary lens surface (as described fully herein) and thereunder are provisioned two separated but longitudinally aligned transducer stacks. In the examination depicted in FIG. 9, the linear array transducer stack is operational and only the subset of the total number of transducer elements dedicated to such an array are activated, and only the first transmit surface is conveying signals/beams, 919. For greater clarity, while the entirety of the imaging end 112 may be depressed against patient P during this examination, only the first transmit surface is conveying signals/beams. Wide frequency ultrasound probe 110 may be wirelessly connected at 920 to interface screen 912 of multi-purpose electronic device 901 wherein an image 922 of carotid artery 924 may be displayed thereon. Like what is shown in FIG. 8, interface screen 912 may comprise controls and guides including but not limited to: mode selector 926, freeze button 928, video icon 930, screen capture icon 932 and depth indicator 934.

It is to be understood, for greater clarity, that in operation, the entirety or substantially entirely of imaging end 112 is depressed against a patient during examination, but only one of the first transmit surface and the second transmit surface is conveying its respective signals/beams and receiving signal back. In some aspects, as an operator moves the probe on/over a surface of the patient, such operator may manually elect to change arrays, for example, by use of inputs on a user interface of a multi-purpose electronic device communicatively connected to the probe or via an operator touching a mode button on the probe itself. In other aspects, the arrays may be switched automatically through elected pre-sets and the like. For Example, a cardiac pre-set may automatically activate the phased array and a vascular pre-set may automatically activate the linear array.

Referring to FIG. 10, shown there is a block diagram of various components of an exemplary dual array probe of the invention generally shown as 1000. There may be a probe body 1010, imaging end 1012, non-imaging end 1014 and middle grip portion 1013 therebetween. Wide frequency probe 1000 may contain a non-transitory computer readable memory 1034 storing computer readable data 1036 and computer readable instructions 1038, which, when executed by the processor 1030, may cause the wide frequency probe 1000 to provide one or more of the functions of the system described herein. Such functions may be, for example, the selection of array to be activated (first array within first transducer stack 1020 or second array within second transducer stack 1022), selection of mode(s) of operation, acquisition of ultrasound data, the processing of ultrasound data, the scan conversion of ultrasound data, the transmission of ultrasound data or ultrasound frames to a display device such as 801 and 901, the detection of operator inputs to the wide frequency probe 1000, and/or the switching of the settings of the wide frequency probe 1000. Selection of operator inputs for functionalities, including selection of mode of operation and/or selection of array to be activated may be by inputs, for example, tactile, verbal and/or other. This may include, for example, buttons such as multi-function buttons on the body of the probe, as shown in FIG. 1, by one or more GUI controls on a multi-functional electronic device (such as shown in FIGS. 8 and 9 as 826 and 926) and/or via audio or text controls to one or more the wide frequency probe and/or the multi-functional electronic device.

The computer readable data 1036 may be used by the processor 1030 in conjunction with the computer readable instructions 1038 to provide the functions of the wide frequency scanner 1000 (including functional control over multi-purpose electronic devices such as 801 and 901, for example, via an application operating on such devices). Computer readable data 1036 may include, for example, configuration settings for wide frequency probe 1000, such as presets that instruct the processor 1030 how to collect and process the ultrasound data and how to acquire a series of ultrasound frames.

Dual array probe 1000 comprises two longitudinally aligned transducer stacks, first transducer stack 1020 and second transducer stack 1022 which each, mutually exclusively, transmit and receive ultrasound energy, at two different frequency ranges, in order to acquire ultrasound frames.

Dual array probe 1000 may include a communications module 1032 connected to the processor 1030. In the illustrated example, the communications module 1032 may wirelessly transmit signals to and receive signals from multi-purpose electronic devices 801 and 901 along wireless communication links, for example 820 and 920. The protocol used for communications between wide frequency probe 1000 and the multi-purpose electronic devices/display devices may be WiFi™ and/or Bluetooth™, for example, or any other suitable two-way radio communications protocol. In some embodiments, wide frequency probe 1000 may operate as a WiFi™ hotspot, for example. Communication links 820 and 920 may use any suitable wireless communications network connection. In some embodiments, the communication link between wide frequency probe 1000 and a selected multi-purpose electronic device/display device may be wired. For example, wide frequency probe 1000 may be attached to a cord that may be pluggable into a physical port of multi-purpose electronic devices/display devices 801 and 901.

In various embodiments, multi-purpose electronic devices/display devices 801 and 901 may be, for example, a laptop computer, a tablet computer, a desktop computer, a smart phone, a smart watch, spectacles with a built-in display, a television, a bespoke display or any other display device that is capable of being communicably connected to dual array probe 1000. Multi-purpose electronic devices/display devices 801 and 901 may host a screen (shown as 812 and 912), and may include a processor, which may be connected to a non-transitory computer readable memory storing computer readable instructions, which, when executed by the processor, cause the display device to provide one or more of the functions of the system (such system comprising at least one multi-purpose electronic device and at least one wide frequency probe of the invention). Such functions may be, for example, the receiving of ultrasound data that may or may not be pre-processed; scan conversion of received ultrasound data into an ultrasound image; processing of ultrasound data in image data frames; the display of a user interface; the control of dual array probe 1000; and the display of an ultrasound image on the screen 812/912. The screen 812/912 may comprise a touch-sensitive display (e.g., touchscreen) that can detect a presence of a touch from the operator on screen 812/912 and can also identify a location of the touch in screen 812/912. The touch may be applied by, for example, at least one of an individual's hand, glove, stylus, or the like. As such, the touch-sensitive display may be used to receive an input, for example, indicating a switch between mutually exclusive activation of the first array and activation of the second array. The screen 812/912 and/or any other user interface may also communicate audibly. Multi-purpose electronic devices/display devices 801 and 901 may be configured to present information to the operator during or after the imaging or data acquiring session. The information presented may include ultrasound images (e.g., one or more 2D frames), graphical elements, measurement graphics of the displayed images, user-selectable elements, user settings, and other information (e.g., administrative information, personal information of the patient, and the like).

Also stored in the computer readable memory within the multi-purpose electronic devices/display devices 801 and 901 may be computer readable data which may be used by processors within multi-purpose electronic devices/display devices 801 and 901, in conjunction with the computer readable instructions within multi-purpose electronic devices/display devices 801 and 901, to provide the functions of the system. Such computer readable data may include, for example, settings for dual array probe 1000, such as presets for acquiring ultrasound data and settings for a user interface displayed on screens 812/912. Settings may also include any other data that is specific to the way that dual array probe 1000 operates or that multi-purpose electronic devices/display devices 801 and 901 operate.

Dual array probe 1000 may be connected via the communications network to a server. The server may itself include a processor, which may be connected to non-transitory computer readable memory storage computer readable instructions, which, when executed by the processor, cause the server to provide one or more of the functions of the system of the invention. Such functions may be, for example, the receiving of ultrasound frames, the processing of ultrasound data in ultrasound frames, the control of dual array probe 1000, the processing of additional inputs related to operation of dual array probe 1000.

It can therefore be understood that the computer readable instructions and data used for controlling the system of the invention may be located either in the computer readable memory 1034, the computer readable memory within multi-purpose electronic devices/display devices 801 and 901, the computer readable memory of the server, or any combination of the foregoing locations.

With reference to both FIGS. 7 and 10, the following system components, including beamformer control, are described. Beamformer control components are responsible for synchronizing the generation of the sound waves and the reflected wave measurements. Such control components know the region of interest in terms of width and depth which is translated into a desired number of scan lines and a desired number of focal points per scan line, for each selected array, at each frequency. Beamformer control begins with a first scan line and excites a selected array of piezo-electric elements (either first array or second array) with a sequence of high voltage pulses via transmit amplifiers. Typical performance numbers for the amplifiers is ±100 V and ±2 Amps for each piezo-electric element. The pulses go through a transmit Tx switch (1026 in FIGS. 10 and 716 in FIG. 7) and a receive Rx switch (1028 in FIGS. 10 and 718 in FIG. 7), which prevents the high voltage pulses from damaging the receive electronics. These high voltage pulses are properly time delayed so that the resulting sound waves can be focused along the desired scan line to produce a narrowly focused beam at a desired focal point. The beamformer control determines (with either a first array or a second array) which transducer elements to energize at a given time and the proper time delay value for each element to properly steer the sound waves towards the desired focal point. As the sound waves propagate toward the desired focal point, they migrate through materials with different densities. With each change in density, the sound wave has a slight change in direction and produces a reflected sound wave. Some of the reflected sound waves propagate back to the transducer and form the input to the piezo-electric elements in the selected array (first array or second array). The resulting low voltage signals are scaled using a variable controlled amplifier (VCA) before being sampled by analog-to-digital converters (ADC), which is herein integrated into Rx switch (1028 in FIGS. 10 and 718 in FIG. 7). The VCA is configured so that the gain profile being applied to the received signal is a function of the sample time since the signal strength decreases with time (e.g., it has traveled through more tissue). The number of VCA and ADC combinations determines the number of active channels used for beamforming. By way of example, it is usual to run the ADC sampling rate 4 times or higher than the transducer center frequency. Once the received signals reach the Rx beamformer, the signals are scaled and appropriately delayed permitting a coherent summation of the signals. This new signal represents the beamformed signal for one or more focal points along a particular specific scan line. The beamformer operations are typically performed in application-specific integrated circuit (ASIC), field-programmable gate array (FPGA), DSP or a combination of these components. The choice of devices depends on the number of channels used in beamforming, which determines the input/output (I/O) requirement as well as the processing requirement to perform digital beamforming.

The manner of manufacturing of the dual array probe of the present invention is not intended to be limited by process steps or materials employed. Notwithstanding the generality of the foregoing, the following provide directives on preferred modes of making the dual transducer stack of the present invention. In regard to the first array comprising the first plurality of transducer elements and the second array comprising the second plurality of transducer elements, direction for manufacturing may be found in the art, as available to skilled parties in the field. Such arrays may be formed of piezoelectric ceramic material such as PZT (Lead Zirconate Titanate). A backing layer/backing block may comprise a predetermined ratio of oxide of metal of, for example, at least one of Cr, Fe, and Cu. It may be formed with a wiring area on at least one side of the backing block whereupon a piezoelectric (array) layer may be placed on the backing block. On the top side of the piezoelectric (array) layer may be attached a matching layer, all as depicted in FIG. 6 and FIG. 11. The backing layer may be formed by mixing a material, for example, polymer resin, having a similar impedance value to an acoustic impedance of the piezoelectric ceramic elements included in the piezoelectric layer and having a high damping coefficient, and a predetermined ratio of oxide of metal of Cr, Fe, Cu, etc. Wherein, in some embodiments, the predetermined ratio may be in a range of more than 0% to less than 21%; however, the ratio is not limited thereto. Hereinafter, the piezoelectric layer may be placed on the backing layer and the matching layer 124 may be placed on the piezoelectric layer, so that each transducer stack can be fabricated. The backing layer, according to one embodiment of the present disclosure, may further include a ground area for connecting with a ground layer. Further, the ground layer in the transducer stack according to one embodiment of the present disclosure is connected to the ground area by using a flexible printed circuit board (FPCB).

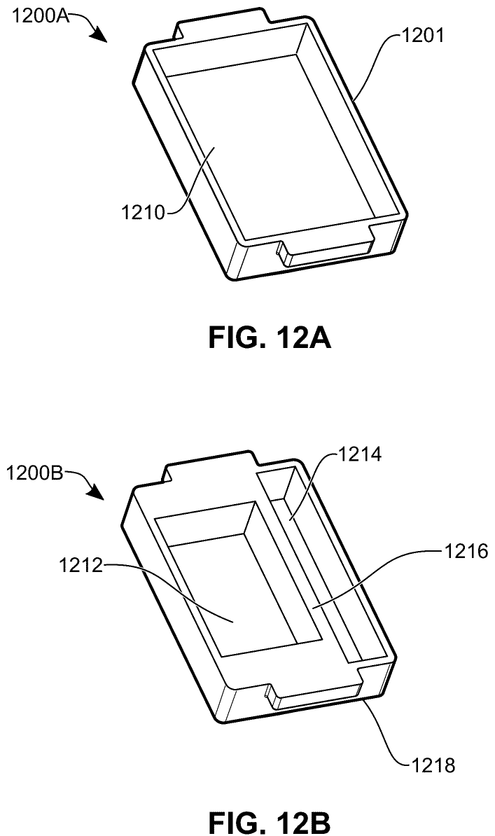

Each of the dual transducer stacks so produced (at least the matching layer, the piezoelectric layer, and the backing layer) are partially encased within an acoustic lens material, e.g., using a sequence of molding as shown in FIGS.12A and B. A lens material is preferably liquid silicone base (PDMS). In FIG. 12A, generally at 1200A, liquid silicone is poured into a mold 1201 forming cavity 1210. At FIG. 12B, generally at 1200B, a pre-formed dual transducer stack assembly (such as shown in FIG. 6) is inserted into mold 1201, and further liquid lens material is poured therearound, leveled and cured. This creates a partial acoustic lens surround, customized to the dual transducer stack assembly and comprising a cavity for phased array 1212, a cavity for linear array 1214, solid lens material filling for colinear space 1216 and outer lens face 1218.

Extending the lens materials in the void around the ‘T’ shape (e.g., to fill in the dummy gaps) increases manufacturability because the mould forms around the T to form a generally box shape, which is easier to remove during manufacturing. In other embodiments, the acoustic lens material can be configured to be just a fixed distance from the transducer stacks (e.g., 1-3 mm) and having the rest of the dummy gaps be filled with another material. This would be less desirable for manufacturability because the acoustic lens mold would also be T-shaped (which is difficult to remove during manufacturing and may result in more damage and lower yield).

### C. Claim Support

The present invention provides, in one aspect, an ultrasound probe comprising: a body comprising an imaging end and a non-imaging end; a first array comprising a first plurality of transducer elements disposed on the imaging end; a second array comprising a second plurality of transducer elements disposed on the imaging end, wherein each the first array and the second array are longitudinally adjacent to each other; a circuit connected to the first plurality of transducer elements and the second plurality of transducer elements; wherein the circuit is capable of activating, in a mutually exclusive manner, the first plurality of transducer elements and the second plurality of transducer elements.

In some embodiments, the probe comprises a unitary lens which covers both the first transmit surface and the second transmit surface. In some embodiments, the imaging end of the probe comprises an imaging contact surface and both the first array and the second array are in substantially the same plane, parallel to the imaging contact surface. In some embodiments, the circuit of the probe is communicatively coupled to a mode selection input, the circuit receiving mode selection input to activate a first mode, in which signals are transmitted only by the first plurality of transducer elements and a second mode, in which signals are transmitted only by the second plurality of transducer elements. In some embodiments, a pitch of the second array of the probe is substantially the same size as the pitch in the first array. In some embodiments, the circuit of the probe is a multiplex circuit and activates the second plurality of transducer elements starting from a start element which is neither 0 nor 1. In some embodiments, the first array of the probe produces a high frequency bandwidth, and the second array of the probe produces a low frequency bandwidth. In some embodiments, the first array of the probe is linear, and the second array of the probe is a phased-array. In some embodiments, the first plurality of transducer elements is greater in number than the second plurality of transducer elements. In some embodiments, there is provisioned a colinear space between the first array and the second array.

The present invention provides, in another aspect, an ultrasound imaging system comprising: an ultrasound probe configured to provide image data; a processor configured to assemble images from said image data; and a display configured to display said images; wherein said ultrasound probe comprises a body comprising an imaging end and a non-imaging end; a first array comprising a first plurality of transducer elements disposed on the imaging end; a second array comprising a second plurality of transducer elements disposed on the imaging end, wherein each the first array and the second array are longitudinally adjacent to each other to each other; wherein the first plurality of transducer elements and the second plurality of transducer elements are connected to a circuit; wherein the circuit is capable of activating, in a mutually exclusive manner, the first plurality of transducer elements and the second plurality of transducer elements.

In some embodiments, within the ultrasound imaging system, the circuit is communicatively coupled to both a mode selection input and to each of the first plurality of transducer elements and the second plurality of transducer elements, the circuit receiving mode selection input to activate a first mode, in which signals are transmitted only by the first plurality of transducer elements and a second mode in which signals are transmitted only by the second plurality of transducer elements. In some embodiments, the first array produces a high frequency bandwidth, and the second array produces a low frequency bandwidth. In some embodiments, the first array is linear and the second array is a phased-array. In some embodiments, a pitch of the second array is substantially the same size as the pitch in the first array. In some embodiments, the first plurality of transducer elements is greater in number than the second plurality of transducer elements.

The present invention provides, in yet another aspect, an ultrasound imaging method comprising, by a wide frequency ultrasound probe: imaging in a first mode using a first array comprising a first plurality of transducer elements disposed on an imaging end of the probe; switching, by a circuit, to a second mode different from the first mode, using a second array comprising a second plurality of transducer elements disposed on the imaging end of the probe; wherein the second array is longitudinally adjacent to the first array; and wherein the circuit is capable of activating, in a mutually exclusive manner, the first plurality of transducer elements and the second plurality of transducer elements.

In some embodiments, within the ultrasound imaging method of the invention, the first array produces a high frequency bandwidth, and the second array produces a low frequency bandwidth. In some embodiments, the first plurality of transducer elements is greater in number than the second plurality of transducer elements. In some embodiments, the first array is linear, and the second array is a phased-array. In some embodiments, a pitch of the second array is substantially the same size as the pitch in the first array. In some embodiments, the circuit is communicatively coupled to both a mode selection input and to each of the first plurality of transducer elements and the second plurality of transducer elements, the circuit receiving mode selection input to activate a first mode, in which signals are transmitted only by the first plurality of transducer elements and a second mode in which signals are transmitted only by the second plurality of transducer elements.

### D. Interpretation of Terms

Unless the context clearly requires otherwise, throughout the description and the claims:
“comprise”, “comprising”, and the like are to be construed in an inclusive sense, as opposed to an exclusive or exhaustive sense; that is to say, in the sense of “including, but not limited to”;
“connected”, “coupled”, or any variant thereof, means any connection or coupling, either direct or indirect, between two or more elements; the coupling or connection between the elements can be physical, logical, or a combination thereof;
“herein”, “above”, “below”, and words of similar import, when used to describe this specification, shall refer to this specification as a whole, and not to any particular portions of this specification;
“or”, in reference to a list of two or more items, covers all of the following interpretations of the word: any of the items in the list, all of the items in the list, and any combination of the items in the list;
the singular forms “a”, “an”, and “the” also include the meaning of any appropriate plural forms.

Unless the context clearly requires otherwise, throughout the description and the claims:

Words that indicate directions such as “vertical”, “transverse”, “horizontal”, “upward”, “downward”, “forward”, “backward”, “inward”, “outward”, “vertical”, “transverse”, “left”, “right”, “front”, “back”, “top”, “bottom”, “below”, “above”, “under”, and the like, used in this description and any accompanying claims (where present), depend on the specific orientation of the apparatus described and illustrated. The subject matter described herein may assume various alternative orientations. Accordingly, these directional terms are not strictly defined and should not be interpreted narrowly.

Embodiments of the invention may be implemented using specifically designed hardware, configurable hardware, programmable data processors configured by the provision of software (which may optionally comprise “firmware”) capable of executing on the data processors, special purpose computers or data processors that are specifically programmed, configured, or constructed to perform one or more steps in a method as explained in detail herein and/or combinations of two or more of these. Examples of specifically designed hardware are: logic circuits, application-specific integrated circuits (“ASICs”), large scale integrated circuits (“LSIs”), very large scale integrated circuits (“VLSIs”), and the like. Examples of configurable hardware are: one or more programmable logic devices such as programmable array logic (“PALs”), programmable logic arrays (“PLAs”), and field programmable gate arrays (“FPGAs”)). Examples of programmable data processors are: microprocessors, digital signal processors (“DSPs”), embedded processors, graphics processors, math co-processors, general purpose computers, server computers, cloud computers, mainframe computers, computer workstations, and the like. For example, one or more data processors in a control circuit for a device may implement methods as described herein by executing software instructions in a program memory accessible to the processors.

For example, while processes or blocks are presented in a given order herein, alternative examples may perform routines having steps, or employ systems having blocks, in a different order, and some processes or blocks may be deleted, moved, added, subdivided, combined, and/or modified to provide alternative or sub combinations. Each of these processes or blocks may be implemented in a variety of different ways. Also, while processes or blocks are at times shown as being performed in series, these processes or blocks may instead be performed in parallel or may be performed at different times.

The invention may also be provided in the form of a program product. The program product may comprise any non-transitory medium which carries a set of computer-readable instructions which, when executed by a data processor (e.g., in a controller and/or ultrasound processor in an ultrasound machine), cause the data processor to execute a method of the invention. Program products according to the invention may be in any of a wide variety of forms. The program product may comprise, for example, non-transitory media such as magnetic data storage media including floppy diskettes, hard disk drives, optical data storage media including CD ROMs, DVDs, electronic data storage media including ROMs, flash RAM, EPROMs, hardwired or preprogrammed chips (e.g., EEPROM semiconductor chips), nanotechnology memory, or the like. The computer-readable signals on the program product may optionally be compressed or encrypted.

Where a component (e.g. a software module, processor, assembly, device, circuit, etc.) is referred to above, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

Specific examples of systems, methods and apparatus have been described herein for purposes of illustration. These are only examples. The technology provided herein can be applied to systems other than the example systems described above. Many alterations, modifications, additions, omissions, and permutations are possible within the practice of this invention. This invention includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

It is therefore intended that the following appended claims and claims hereafter introduced are interpreted to include all such modifications, permutations, additions, omissions, and sub-combinations as may reasonably be inferred. The scope of the claims should not be limited by the preferred embodiments set forth in the examples but should be given the broadest interpretation consistent with the description as a whole.

What is claimed is:

1. An ultrasound probe comprising:
a body comprising an imaging end and a non-imaging end;
a first array comprising a first plurality of transducer elements disposed on the imaging end;
a second array comprising a second plurality of transducer elements disposed on the imaging end, wherein each the first array and the second array are longitudinally adjacent to each other;
a circuit connected to the first plurality of transducer elements and the second plurality of transducer elements;
wherein the circuit is capable of activating, in a mutually exclusive manner, the first plurality of transducer elements and the second plurality of transducer elements.

2. The ultrasound probe of claim 1 comprising a unitary lens which covers both the first transmit surface and the second transmit surface.

3. The ultrasound probe of claim 1 wherein the imaging end comprises an imaging contact surface and both the first array and the second array are in substantially the same plane, parallel to the imaging contact surface.

4. The ultrasound probe of claim 1 wherein the circuit is communicatively coupled to a mode selection input, the circuit receiving mode selection input to activate a first mode, in which signals are transmitted only by the first plurality of transducer elements and a second mode, in which signals are transmitted only by the second plurality of transducer elements.

5. The ultrasound probe of claim 1 wherein a pitch of the second array is substantially the same size as the pitch in the first array.

6. The ultrasound probe of claim 1 wherein the circuit is a multiplex circuit and activates the second plurality of transducer elements starting from a start element which is neither 0 nor 1.

7. The ultrasound probe of claim 1 wherein the first array produces a high frequency bandwidth and the second array produces a low frequency bandwidth.

8. The ultrasound probe of claim 1 wherein the first array is linear and the second array is a phased-array.

9. The ultrasound probe of claim 1 wherein the first plurality of transducer elements is greater in number than the second plurality of transducer elements.

10. The ultrasound probe of claim 1 comprising a colinear space between the first array and the second array.

11. An ultrasound imaging system comprising:
an ultrasound probe configured to provide image data;
a processor configured to assemble images from said image data; and
a display configured to display said images;
wherein said ultrasound probe comprises:
a body comprising an imaging end and a non-imaging end;
a first array comprising a first plurality of transducer elements disposed on the imaging end;
a second array comprising a second plurality of transducer elements disposed on the imaging end, wherein each the first array and the second array are longitudinally adjacent to each other to each other;
wherein the first plurality of transducer elements and the second plurality of transducer elements are connected to a circuit;
wherein the circuit is capable of activating, in a mutually exclusive manner, the first plurality of transducer elements and the second plurality of transducer elements.

12. The ultrasound imaging system of claim 11 wherein the circuit is communicatively coupled to both a mode selection input and to each of the first plurality of transducer elements and the second plurality of transducer elements, the circuit receiving mode selection input to activate a first mode, in which signals are transmitted only by the first plurality of transducer elements and a second mode in which signals are transmitted only by the second plurality of transducer elements.

13. The ultrasound imaging system of claim 11 wherein the first array produces a high frequency bandwidth and the second array produces a low frequency bandwidth.

14. The ultrasound imaging system of claim 11 wherein the first array is linear and the second array is a phased-array.

15. The ultrasound imaging system of claim 11 wherein a pitch of the second array is substantially the same size as the pitch in the first array.

16. The ultrasound imaging system of claim 11 wherein the first plurality of transducer elements is greater in number than the second plurality of transducer elements.

17. An ultrasound imaging method comprising, by a wide frequency ultrasound probe:
imaging in a first mode using a first array comprising a first plurality of transducer elements disposed on an imaging end of the probe;
switching, by a circuit, to a second mode different from the first mode, using a second array comprising a second plurality of transducer elements disposed on the imaging end of the probe;
wherein the second array is longitudinally adjacent to the first array;
and wherein the circuit is capable of activating, in a mutually exclusive manner, the first plurality of transducer elements and the second plurality of transducer elements.

18. The ultrasound imaging method of claim 17 wherein the first array produces a high frequency bandwidth, and the second array produces a low frequency bandwidth, and the first plurality of transducer elements is greater in number than the second plurality of transducer elements.

19. The ultrasound imaging method of claim 17 wherein the first array is linear, and the second array is a phased-array, and a pitch of the second array is substantially the same size as the pitch in the first array.

20. The ultrasound imaging method of claim 17 wherein the circuit is communicatively coupled to both a mode selection input and to each of the first plurality of transducer elements and the second plurality of transducer elements, the circuit receiving mode selection input to activate a first mode, in which signals are transmitted only by the first plurality of transducer elements and a second mode in which signals are transmitted only by the second plurality of transducer elements.
